(19) United States
(12) Patent Application Publication
(10) Pub. No.: US 2014/0058243 A1
(43) Pub. Date: Feb. 27, 2014

(54) BIOSENSOR ELECTRODE DEVICE AND METHOD FOR FABRICATING THE SAME

(71) Applicant: NATIONAL CHIAO TUNG UNIVERSITY, Hsinchu City (TW)

(72) Inventors: CHIN-TENG LIN, HSINCHU COUNTY (TW); LUN-DE LIAO, TAICHUNG CITY (TW)

(73) Assignee: NATIONAL CHIAO TUNG UNIVERSITY, HSINCHU CITY (TW)

(21) Appl. No.: 13/736,153

(22) Filed: Jan. 8, 2013

(30) Foreign Application Priority Data
Aug. 23, 2012 (TW) 101130559

(51) Int. Cl. A61B 5/04

(52) U.S. Cl. CPC A61B 5/04
USPC 600/396; 600/372; 29/876

(57) ABSTRACT

A biosensor electrode device and a method for fabricating the same are disclosed. The biosensor electrode device comprises a conductive substrate; at least one conductive spring, at least one probe, and an encapsulation member. Two ends of the conductive spring respectively connect with the conductive substrate and the probe. The probe moves close to or far away from the substrate through the elastic deformation of the conductive spring. The encapsulation member wraps the conductive substrate, the conductive springs and the probes but reveals a portion of each probe. The revealed probes contact skin of a testee for biomedical measurement. The device and method of the present invention can reduce the cost in mass-production. The biosensor electrode device will be a mainstream instrument in biomedical measurement.

### BACKGROUND OF THE INVENTION

1. Field of the Invention

The present invention relates to an electrode technology, particularly to a biosensor electrode device and a method for fabricating the same.

2. Description of the Related Art

Biomedical measurement systems are popular medial apparatuses. Many research papers have been proposed to improve the inconveniences and drawbacks of biomedical measurement systems. Some of them even intend to integrate biosensors with pioneering technologies for military, biomedical and HMI (Human-Machine Interaction) applications.

Traditional EEG (electroencephalography) systems normally use wet electrodes. Recently, dry electrodes are frequently mentioned. Dry electrodes are more convenient than wet electrodes because wet electrodes have to cooperate with conductive glue. Conductive glue may cause discomfort or allergy to patients. Besides, conductivity of conductive glue decays with time. Similar to the electrodes of EEG systems, the electrodes of biomedical measurement systems also need conductive glue and have the common drawbacks of wet electrodes.

At present, dry electrodes are almost fabricated in microstructure processes, such as the MEMS (microelectromechanical system) process and the carbon nanotube process. However, the microstructure-based dry electrodes are likely to fracture and hard to apply to hairy regions. Therefore is impaired popularization of dry electrodes.

Biomedical science has attracted more and more attention in recent years. Therefore, improvement and application of biomedical measurement apparatuses become an important subject. The current tendency is to miniaturize apparatuses and realize instant and long-term biomedical measurement. The conventional bulky and complicated devices have been out of date. However, many researches are still impeded by shortage of efficacious and cost-efficient technologies.

### SUMMARY OF THE INVENTION

The primary objective of the present invention is to provide a biosensor electrode device and a method for fabricating the same, wherein the electrode device of the present invention is exempted from involving with the conductive glue and therefore avoiding the drawbacks of the conventional wet electrodes.

Another objective of the present invention is to provide a biosensor electrode device and a method for fabricating the same, wherein an injection molding process is used to fabricate a novel electrode device, which applies to measuring biomedical signals, such as EEG, ECG, EMG and EOG, and will function as a mainstream instrument in biomedical measurement.

A further objective of the present invention is to provide a biosensor electrode device and a method for fabricating the same, wherein the high-conductivity electrodes directly contact the skin and detect very weak signals with a very high sensitivity without using conductive glue, whereby the operation is more convenient and efficient.

To achieve the abovementioned objectives, the present invention proposes a biosensor electrode device, which applies to detecting biomedical signals of a testee, and which comprises a conductive substrate; at least one conductive spring having a first end and a second end, wherein the first end connects with the conductive substrate; at least one probe connecting with the second end of the conductive spring and moved close to or far away from the conductive substrate through the elastic deformation of the conductive spring; and an encapsulation member wrapping the conductive substrate, the conductive spring and a portion of each probe but revealing the other portion of each probe. The at least one probe of the biomedical measurement electrode device contacts the skin of the testee for biomedical measurement.

In one embodiment, the conductive substrate is made of a flexible material.

In one embodiment, the probe is made of gold or silver chloride. In one embodiment, the probe has a diameter of greater than 1.3 mm. In one embodiment, the distribution density of the probes approximates to the skin capillary density. In one embodiment, the probes are staggered from the skin capillaries lest they coincide.

In one embodiment, the biosensor electrode device has a plurality of conductive springs and a plurality of probes, wherein a second end of each conductive spring connects with one probe, and wherein first ends of all the conductive springs connect with the conductive substrate, and wherein the probes are moved close to or far away from the conductive substrate through the elastic deformation of the conductive springs.

The present invention also proposes a method for fabricating a biosensor electrode device, which comprises steps: providing at least one conductive spring having a first end and a second end; connecting the first end of the conductive spring with a conductive substrate; providing at least one probe connecting with the second end of the conductive spring such that the probe moves close to or far away from the conductive substrate through the elastic deformation of the conductive spring; using an encapsulation member to wrap the conductive substrate, the conductive springs and a portion of each probe but reveal the other portion of each probe, which is to contact the skin of the testee for biomedical measurement.

In one embodiment, the method further comprises a step of arranging an anti-electrostatic and anti-electromagnetic shell outside the encapsulation member.

Below, embodiments are described in detail in cooperation with drawings to make easily understood the objectives, technical contents, characteristics and accomplishments of the present invention.

### BRIEF DESCRIPTION OF THE DRAWINGS

FIG. 1A is a bottom view schematically showing a biosensor electrode device according to one embodiment of the present invention;

FIG. 1B is a side view schematically showing a biosensor electrode device according to one embodiment of the present invention;

FIG. 2 shows a flowchart of a method for fabricating a biosensor electrode device according to one embodiment of the present invention;

FIG. 3 schematically shows the internal structure of a biosensor electrode device according to one embodiment of the present invention;

FIG. 4 schematically shows the internal structure of a biosensor electrode device according to another embodiment of the present invention;

FIG. 5 shows a flowchart of a method for fabricating a biosensor electrode device having a shell according to one embodiment of the present invention;

FIG. 6 is a sectional view schematically showing a biosensor electrode device having a shell according to one embodiment of the present invention; and

FIG. 7 is a sectional view schematically showing a biosensor electrode device having a flexible layer according to one embodiment of the present invention.

### DETAILED DESCRIPTION OF THE INVENTION

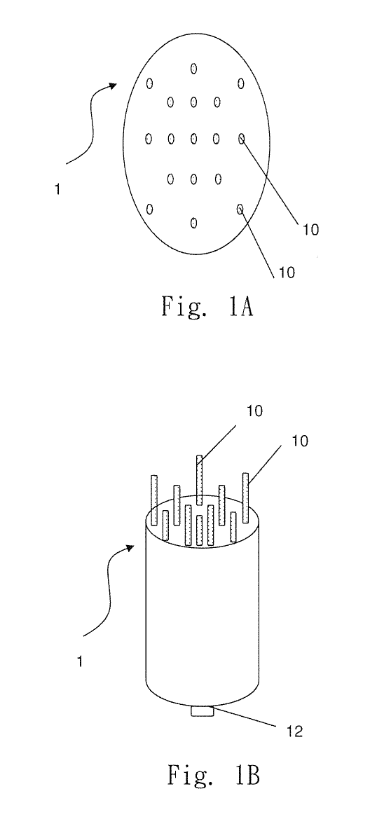

Refer to FIG. 1A and FIG. 1B respectively showing a bottom view and a side view of a biosensor electrode device according to one embodiment of the present invention.

The biosensor electrode device 1 of the present invention has a plurality of probes 10 for contacting the skin of the testee to implement biomedical measurements, such as EEG (electroencephalography), ECG (electrocardiography), EMG (electromyography) and EOG (electrooculography) measurements.

The biosensor electrode device 1 has a female fastener 12, via which the biosensor electrode device 1 is fastened to a biosensing system for medical specialists to operate.

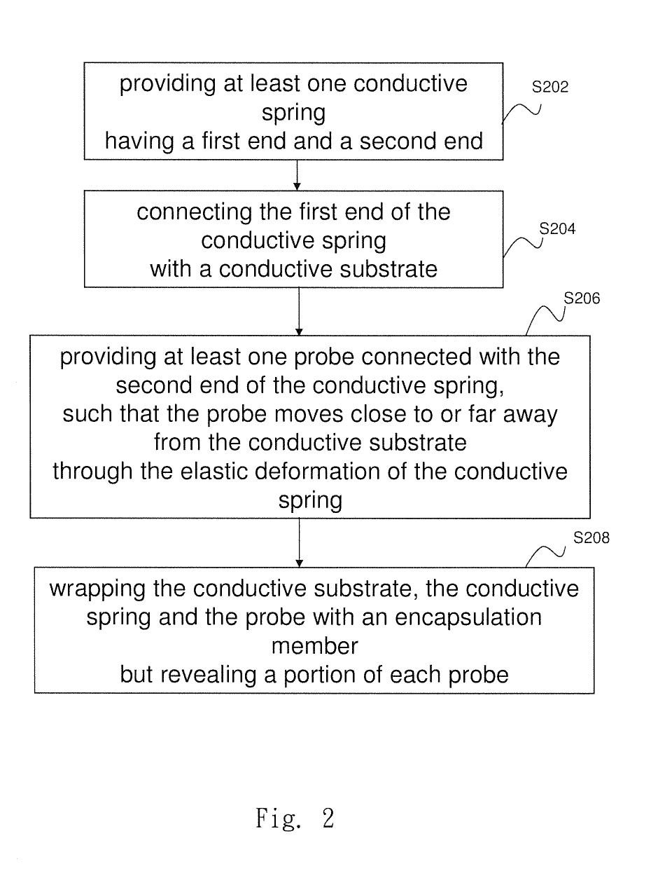

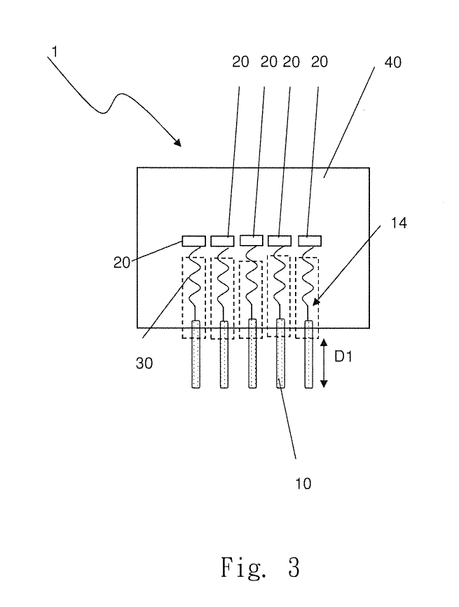

Refer to FIG. 2, which is a flowchart of a method for fabricating a biosensor electrode device according to one embodiment of the present invention. Also refer to FIG. 3 schematically showing the internal structure of a biosensor electrode device according to one embodiment of the present invention. The method of the present invention comprises Steps S202, S204, S206 and S208.

Firstly, in Step S202, provide at least one conductive spring 30 each having a first end and a second end.

Next, in Step S204, connect the first end of each conductive spring 30 with a conductive substrate 20.

Next, in Step S206, provide at least one probe 10 each connecting with the second end of one conductive spring 30 such that the probes 10 move close to or far away from the conductive substrate 20 through the elastic deformation of the conductive spring to generate a displacement D1.

As shown in FIG. 3, each conductive spring 30 and the probe 10 connected thereto are arranged inside a tube 14, and the interior of the tube 14 is in a vacuum state.

Next, in Step S208, use an encapsulation member 40 to wrap the conductive substrate 20, the conductive springs 30 and a portion of each probe 10 but reveal the other portion of each probe 10, whereby the revealed probes 10 can contact the skin of the testee for biomedical measurement.

In one embodiment, the conductive substrate 20 is a printed circuit board or a metal plate. In one embodiment, the conductive substrate 20 is made of a flexible material. Therefore, the probes 10 and the conductive springs 30 can be embedded in the conductive substrate 20 in a stamping way. Because of flexibility, the conductive substrate 20 can deform slightly to conform to the topography of the testee's skin. Thereby, each probe 10 can output the signals of a single point via the conductive substrate 20 connected to an external circuit.

In the present invention, the probes 10 are made of a biocompatible conductive material, such as gold (Au) or silver chloride (AgCl). Similarly to the probes for IC test, one embodiment of the present invention adopts a high-conductivity metal as the material of the probes 10. A gold layer is further coated on the surfaces of the probes 10, enhancing electric conduction and preventing the skin from allergy. As the ends of the probes 10 are joined with the conductive springs 30, the elastic deformation of the conductive springs 10 enables the probes 10 to move close to or far away from the conductive substrate 20 and perfectly conform to the topography of the testee's skin.

In practical application, the probes 10 are very small and numerous. Therefore, the biosensor electrode device can still work well in the thick-haired regions. In one embodiment, the probe 10 has a diameter of 1.3 mm (about the diameter of a skin capillary); the distribution density of the probes 10 approximate to the distribution density of the skin capillaries; and the probes 10 are staggered from the skin capillaries lest the probes 10 be inserted into the skin capillaries and wound the skin.

In one embodiment, the encapsulation member 40 is made of plastic, acrylic, silicone, or rubber. In a preferred embodiment, the encapsulation member 40 is fabricated with silicone in an injection-molding method, wrapping the whole conductive substrate 20 and the roots of the probes 10.

In one embodiment, the silicone component (the encapsulation member 40) is fabricated in an injection-molding method, wherein the assembled probes 10, conductive springs 30 and the conductive substrate 20 are placed in a mold and silicone are injected into the mold to fully wrap the abovementioned elements. Since silicone is a soft material, the soft encapsulation member 40, together with and the flexible conductive substrate 20, enables the biosensor electrode device 1 to conform to the topography of the testee's skin. Thereby is improved the accuracy of biomedical measurement. In addition to the conductive substrate 20 and the encapsulation member 40, the metallic probes 10 are also high flexible. Therefore, the biosensor electrode device 1 can still securely adhere to the tested region in sport.

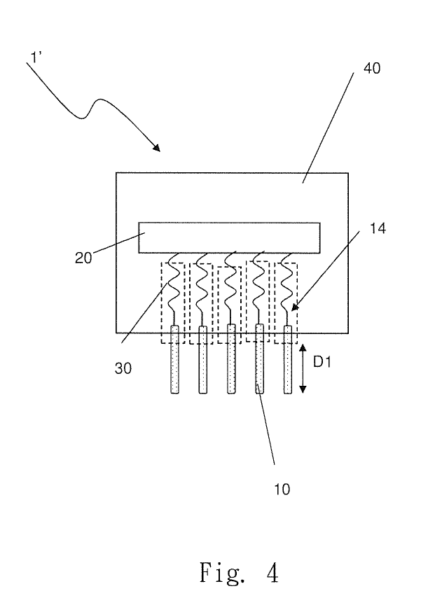

In another embodiment, the biosensor electrode device 1′ of the present invention may further include a plurality of conductive springs 30 and a plurality of probes 10, and the first ends of all the conductive spring 30 are jointly connected to a single conductive substrate 20, as shown in FIG. 4.

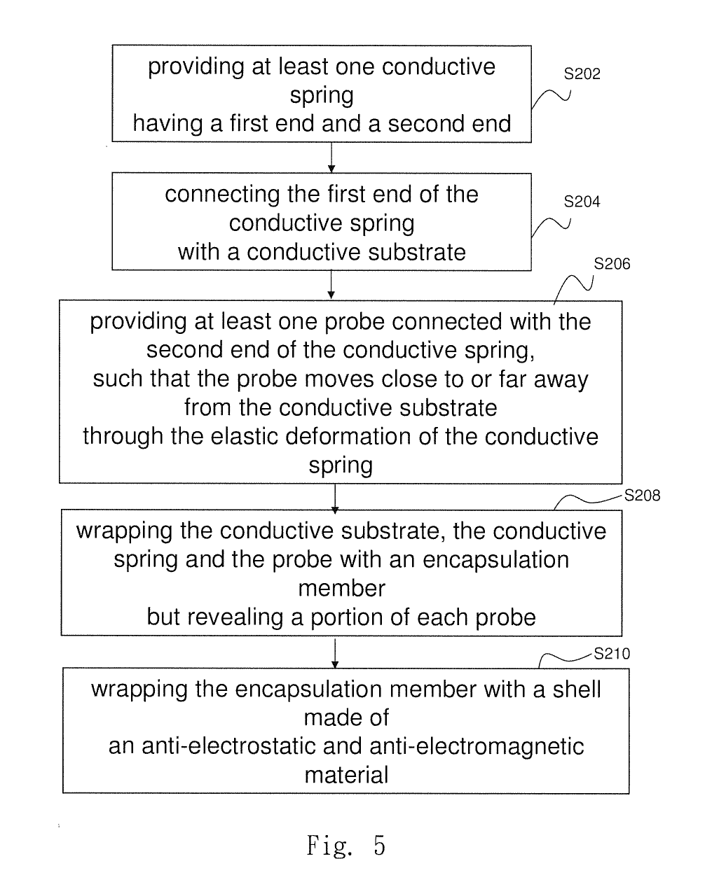

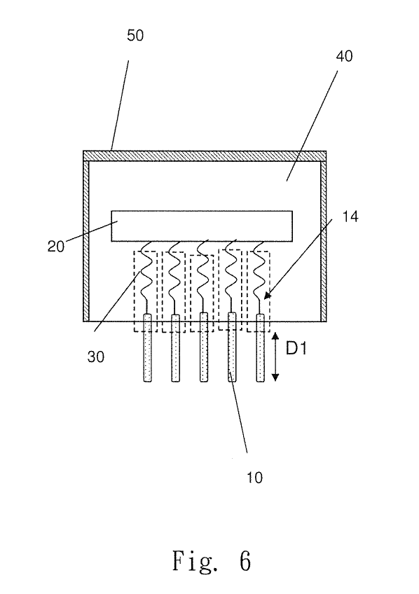

Moreover, the biosensor electrode device 1 of the present invention may further comprise an anti-electrostatic and anti-electromagnetic shell 50 wrapping the encapsulation member 40, as shown in FIG. 6. In this embodiment, the fabricating method further comprises Step S210 to fabricate the anti-electrostatic and anti-electromagnetic shell 50, as shown in FIG. 5. Thus is completed the biosensor electrode device 1 of the present invention. Then, a female fastener 12 (shown in FIG. 1B) is used to fasten the biosensor electrode device 1 to a biosensor for biomedical measurement.

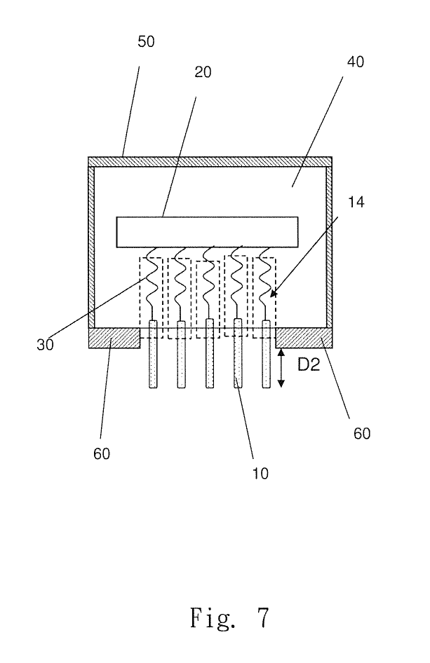

In order to increase the flexibility of the biosensor electrode device and the comfort of the testees, the present invention further comprises a flexible layer 60 arranged on the bottom of the encapsulation member 40, as shown in FIG. 7. The probes 10 normally slightly protrude from the encapsulation member 40. While the probes 10 withdraw toward the conductive substrate 20, the testee's skin would not contact the tubes 14 but will contact the flexible layer 60. In such a case, the probes 10 are constrained by the flexible layer 60 to withdraw for a displacement of D2, which is smaller than D1.

To sum up, the conventional biomedical measurement technology often needs as many as wet electrodes and each of them is very different in conductivity more or less. Therefore, the conventional biomedical measurement technology spends much time in preparing and calibrating the electrodes and biosensor. The MEMS electrodes cannot apply to the thick-haired regions. However, the biosensor electrodes of the present invention not only have superior conductivity but also can work instantly after directly contacting the skin. In comparison with the conventional technologies, the present invention has the advantages of operation convenience and measurement precision.

Besides, the method of the present invention adopts a technology, which has never applied to the like products. Further, the method of the present invention adopts flexible silicone as the material of the encapsulation member to enable the electrode device to conform to the topography of the testee's skin.

Furthermore, the probes of the present invention are disposable. The probes and conductive substrate can be dismounted from the anti-electrostatic and anti-electromagnetic shell and replaced for different testees. The probes and conductive substrate of the present invention can be fabricated with a traditional technology. Therefore, the price thereof can be reduced in mass-production. All the abovementioned advantages would make the biosensor electrode device of the present invention one of the mainstream instruments in biomedical measurement.

The embodiments described abovementioned are only to exemplify the present invention to enable the persons skilled in the art to understand, make, and use the present invention. However, it is not intended to limit the scope of the present invention. Any equivalent modification or variation according to the spirit of the present invention is to be also included within the scope of the present invention.

What is claimed is:

1. A biosensor electrode device, which applies to measuring biomedical signals of a testee, comprising
a conductive substrate;
at least one conductive spring having a first end and a second end, wherein said first end is connected with said conductive substrate;
at least one probe, for contacting skin of said testee for measuring biomedical signals, wherein said at least one probe is connected with said second end of said at least one conductive spring, and moves close to or far away from said conductive substrate through elastic deformation of said at least one conductive spring; and
an encapsulation member wrapping said conductive substrate, said at least one conductive spring and said at least one probe but revealing a portion of each said probe.

2. The biosensor electrode device according to claim 1, wherein said conductive substrate is a printed circuit board or a metal plate.

3. The biosensor electrode device according to claim 1, wherein said conductive substrate is made of a flexible material.

4. The biosensor electrode device according to claim 1, wherein said encapsulation member is made of plastic, acrylic, silicone, or rubber.

5. The biosensor electrode device according to claim 1, wherein said probe is made of gold or silver chloride.

6. The biosensor electrode device according to claim 1, wherein said probe has a diameter of greater than 1.3 mm.

7. The biosensor electrode device according to claim 1 comprising a plurality of said conductive springs and a plurality of said probes, wherein said second ends of said conductive springs are respectively connected with said probes, and wherein said first ends of said conductive springs are jointly connected with said conductive substrate, and wherein said probes move close to or far away from said conductive substrate through elastic deformation of said conductive springs.

8. The biosensor electrode device according to claim 7, wherein distribution density of said probes approximates to distribution density of skin capillaries of said testee.

9. The biosensor electrode device according to claim 7, wherein said probes are staggered with respect to skin capillaries of said testee.

10. The biosensor electrode device according to claim 1, wherein said conductive spring and said probe connected with said conductive spring are arranged in a tube, and wherein interior of said tube is in a vacuum state.

11. The biosensor electrode device according to claim 10, wherein a flexible layer is arranged outside said encapsulation member, and wherein while said probe withdraws toward to said conductive substrate, skin of said testee does not contact said tube but contacts said flexible layer.

12. The biosensor electrode device according to claim 1 further comprising a shell wrapping said encapsulation member, wherein said shell is made of an anti-electrostatic and anti-electromagnetic material.

13. A method for fabricating a biosensor electrode device, comprising steps:
providing at least one conductive spring having a first end and a second end;
connecting said first end of said at least one conductive spring with a conductive substrate;
providing at least one probe for contacting skin of a testee for measuring biomedical signals, wherein said at least one probe is connected with said second end of said at least one conductive spring, and moves close to or far away from said conductive substrate through elastic deformation of said at least one conductive spring; and
wrapping said conductive substrate, said at least one conductive spring and said at least one probe with an encapsulation member but revealing a portion of each said probe.

14. The method for fabricating a biosensor electrode device according to claim 13, wherein said conductive substrate is made of a flexible material.

15. The method for fabricating a biosensor electrode device according to claim 13, wherein said probe is made of gold or silver chloride.

16. The method for fabricating a biosensor electrode device according to claim 13 further comprising a step:
wrapping said encapsulation member with a shell made of an anti-electrostatic and anti-electromagnetic material.

17. The method for fabricating a biosensor electrode device according to claim 13, wherein said encapsulation member is fabricated with an injection-molding method.
